(19) United States
(12) Patent Application Publication
(10) Pub. No.: US 2023/0190734 A1
(43) Pub. Date: Jun. 22, 2023

(54) LEVOCETIRIZINE AND MONTELUKAST IN THE TREATMENT OF CORONAVIRUS DISEASE AND SYMPTOMS THEREOF

(71) Applicant: IRR, Inc., Santa Barbara, CA (US)

(72) Inventor: Bruce Chandler May, Santa Barbara, CA (US)

(21) Appl. No.: 18/082,353

(22) Filed: Dec. 15, 2022

Related U.S. Application Data
(60) Provisional application No. 63/290,285, filed on Dec. 16, 2021.

(51) Int. Cl. A61K 31/495; A61K 31/47; A61P 31/14; A61K 45/06

(52) U.S. Cl. CPC A61K 31/495; A61K 31/47; A61P 31/14; A61K 45/06

(57) ABSTRACT

Certain embodiments described herein include methods and formulations for treating or preventing symptoms and conditions associated with coronavirus disease (e.g., COVID-19). The methods and formulations include, but are not limited to, methods and formulations for delivering effective concentrations of levocetirizine and montelukast to a patient in need. The methods and formulations can comprise conventional and/or modified-release elements, providing for drug delivery to the patient.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The disclosure generally relates to treating coronavirus disease (covid, e.g., COVID-19, SARS-CoV-2) in patients with the combination of levocetirizine and montelukast.

Coronavirus disease (COVID-19) is an infectious disease caused by the SARS-CoV-2 virus. Most people infected with the virus will experience mild to moderate respiratory illness and recover without requiring special treatment. However, some will become seriously ill and require medical attention. Older people and those with underlying medical conditions like cardiovascular disease, diabetes, chronic respiratory disease, or cancer are more likely to develop serious illness. Anyone can get sick with COVID-19 and become seriously ill or die at any age.

### SUMMARY

Embodiments of compositions and methods for treating a patient suffering from coronavirus disease (covid, e.g., COVID-19, SARS-CoV-2) or a symptom thereof are disclosed. In several embodiments, coronavirus disease includes variants of COVID-19. In several embodiments, the compositions are suitable for treating a patient suffering from a COVID-19 variant, including but not limited to Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), Delta (B.1.617.2 and AY lineages), Omicron (B.1.1.529 and BA lineages), and/or mutant descendants of any of the foregoing (or other variants of COVID-19, such as variants of concern, variants of interest, etc.). In some embodiments, the composition comprises an effective amount of a combination of levocetirizine and montelukast. In some embodiments, the method comprises administering to the patient an effective amount of a combination of levocetirizine and montelukast. In some embodiments, the treatment causes a decrease in severity of the signs (objective or subjective) or symptoms (subjective or objective) of coronavirus disease (e.g., COVID-19, SARS-CoV-2, or variants thereof) including but not limited to one or more of fever, chills, cough, shortness of breath, difficulty breathing, fatigue, muscle aches, body aches, headache, new loss of taste or smell, sore throat, congestion, runny nose, nausea, vomiting, diarrhea, mental confusion, headache, chills, rapid heart rate, and/or rapid breathing.

In some embodiments, the combination of levocetirizine and montelukast is administered in a sequential manner. In some embodiments, the combination of levocetirizine and montelukast is administered in a substantially simultaneous manner.

In some embodiments, the combination is administered to the patient by one or more of the routes consisting of enteral, intravenous, intraperitoneal, inhalation, intramuscular, subcutaneous and oral. In some embodiments, the levocetirizine and montelukast are administered by the same route. In some embodiments, the levocetirizine and montelukast are administered via different routes. In some embodiments, one or more of levocetirizine or montelukast are provided as a slow release composition.

In some embodiments, the combination further comprises other medications known for use in treating coronavirus disease (e.g., COVID-19, SARS-CoV-2) and/or complications associated with coronavirus disease (e.g., COVID-19, SARS-CoV-2). In some embodiments, the combination further comprises one or more of antipyretics, analgesics, antitussives, anti-SARS-CoV-2 monoclonal antibody (mAb) products (bamlanivimab plus etesevimab; casirivimab plus imdevimab; a single mAb sotrovimab, etc.), dexamethasone or other systemic glucocorticoids, Remdesivir, baricitinib, and/or combinations of any of the foregoing. In some embodiments, the combination further comprises a steroid.

Some embodiments pertain to a composition for use in treating a patient having coronavirus disease, the composition comprising a combination of levocetirizine and montelukast.

### DETAILED DESCRIPTION

Some examples described herein disclose methods for using levocetirizine and montelukast as a medicament for the prevention or treatment of coronavirus disease and/or complications of coronavirus disease and/or damage caused by coronavirus disease. Some embodiments pertain to compositions comprising levocetirizine and montelukast for use in treating coronavirus disease. Some embodiments provide dosing regimens of levocetirizine and montelukast for treating patients with coronavirus disease. In several embodiments, levocetirizine and montelukast may be used in combination with another coronavirus treatment. The examples described herein are illustrative and not intended in any way to restrict the general inventions presented and the various aspects and features of these inventions. Furthermore, the phraseology and terminology used herein is for the purpose of description and should not be regarded as limiting. No features or steps disclosed herein are essential or indispensable.

As used herein, “treat,” “treatment,” “treating,” “ameliorate,” “amelioration,” “ameliorating,” “improvement,” or “improving” refers to reducing, and/or alleviating the acute and/or long-term effects of a coronavirus disease (increasing reducing the incidences of death). Treatment may comprise one or more of slowing progression, shortening duration, alleviating and/or reducing symptoms (or complications), alleviating and/or reducing associated secondary conditions, decreasing the duration of symptoms, decreasing the duration of associated secondary conditions, and/or alleviating or decreasing long term or residual effects and/or associated secondary issues associated with coronavirus disease. In some embodiments, “treating,” (or “treatment”) “ameliorating,” (or “ameliorate”) and/or “improving” (or “improvement”) refers to a detectable improvement and/or a detectable change consistent with improvement that occurs in a subject or in at least a minority of subjects, e.g., in at least about: 2%, 5%, 10%, 15%, 20%, 25%, 30%, 40%, 50%, 60%, 70%, 75%, 80%, 85%, 90%, 95%, 98%, 100%, or ranges including and/or spanning the aforementioned values. In some embodiments, “treating,” “ameliorating,” and/or “improving” coronavirus disease refers to lowering the severity of signs/symptoms associated with coronavirus disease. In some embodiments, such improvement or change may be observed in treated subjects as compared to subjects not treated with levocetirizine and montelukast, where the untreated subjects have been exposed to the same source of infection, are suffering from the same or a similar severity of coronavirus disease, or are subject to developing the same or similar disease condition, symptom, or the like. In some embodiments, treatment of a disease state (e.g., coronavirus disease), condition, symptom or assay parameter may be determined subjectively or objectively, e.g., by in vitro assays, self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s), by detection of respiratory or inflammatory disorders in a subject, detection of fever, detection of degree of organ failure, detection of degree of tissue damage, and/or by modalities such as, but not limited to photographs, video, digital imaging, endoscopy, biopsy, and pulmonary function tests. Treatment may be transient, prolonged or permanent and/or it may be variable at relevant times during or after levocetirizine and montelukast are administered to a subject. Treatment with levocetirizine and montelukast may be evident from an assay (e.g., an in vitro assay, an in vivo assay, etc.). In some embodiments, the levocetirizine and montelukast treatment is curative. In some embodiments, the levocetirizine and montelukast combination successfully treats a patient when the combination is administered within timeframes described infra, or when administration occurs about 1 hour after, 1 day after, 1 week after the subject(s) has first shown a sign or symptom of coronavirus disease infection. In some embodiments, the levocetirizine and montelukast treatment is preventative. In some embodiments, the levocetirizine and montelukast combination successfully treats a patient when the combination is administered within timeframes described infra, or when viral exposure occurs about 1 hour after the administration or use of levocetirizine and montelukast to about 1 day, or 2, 3, 6, 9 days or more after a subject(s) has received such treatment (e.g., prophylactic use).

The “modulation” of, e.g., a symptom or condition, level or biological activity of a molecule, or the like, refers, for example, to the symptom or activity, or the like that is detectably increased or decreased. Such increase or decrease may be observed in treated subjects as compared to subjects not treated with levocetirizine and montelukast, where the untreated subjects have, or are subject to developing, the same or similar disease state, condition, symptom, complication, or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or ranges including and/or spanning the aforementioned values. Modulation may be determined subjectively or objectively, e.g., by the subject's self-assessment, by a clinician's assessment or by conducting an appropriate assay or measurement, including, e.g., quality of life assessments, suitable assays for the level or activity of molecules, cells or cell migration within a subject and/or by modalities such as, but not limited to photographs, video, digital imaging, X-ray, biopsy, and pulmonary function tests. Modulation may be transient, prolonged or permanent or it may be variable at relevant times during or after levocetirizine and montelukast are administered to a subject or is used in an assay or other method described herein or a cited reference, e.g., within times described infra.

As used herein, the terms “prevent,” “preventing,” and “prevention” refer to the prevention of onset or development of damage associated with or caused by coronavirus disease and or microbial infection that is likely to result in coronavirus disease. Preventing includes protecting against the occurrence and lowering the severity of damage associated with coronavirus disease.

As used herein, the terms “complications associated with coronavirus disease” include, but are not limited to, symptoms and secondary conditions associated with exposure to infection from coronavirus, including loss of lung function, pneumonia, acute respiratory distress syndrome (ARDS), multi-organ failure, septic shock, and death.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

As disclosed elsewhere herein, some embodiments disclosed herein provide methods for using levocetirizine and montelukast as a medicament for the prevention or treatment of coronavirus disease, complications associated with coronavirus disease, and/or damage caused by coronavirus disease. Some embodiments pertain to compositions comprising levocetirizine and montelukast for use in treating coronavirus disease. Some embodiments provide dosing regimens of levocetirizine and montelukast for treating patients with coronavirus disease.

In several embodiments, candidates for treatment (e.g., patients) are selected. In several embodiments, to a candidate for treatment is administered levocetirizine and montelukast (e.g., a composition comprising levocetirizine and montelukast). In some embodiments, candidates for treatment in the disclosed methods include patients suffering from or at risk of suffering from coronavirus infection (and/or coronavirus disease), including patients at risk for severe coronavirus disease. Patients at risk for severe disease may be selected for treatment. Patients at risk for severe disease may include those who are older adults are more likely to get severely ill from COVID-19. More than 81% of COVID-19 deaths occur in people over age 65. The number of deaths among people over age 65 is 80 times higher than the number of deaths among people aged 18-29. Patients at risk for severe disease may include those having underlying medical conditions (e.g., cancer, chronic kidney disease, chronic liver disease, chronic lung disease (e.g., Asthma, if it's moderate to severe; Bronchiectasis (thickening of the lungs airways); Bronchopulmonary dysplasia (chronic lung disease affecting newborns); Chronic obstructive pulmonary disease (COPD), including emphysema and chronic bronchitis; Having damaged or scarred lung tissue such as interstitial lung disease (including idiopathic pulmonary fibrosis); Cystic fibrosis, with or without lung or other solid organ transplant; Pulmonary embolism (blood clot in the lungs); Pulmonary hypertension (high blood pressure in the lungs)), dementia or other neurological conditions, diabetes (type 1 or type 2), down syndrome, heart conditions (heart failure, coronary artery disease, cardiomyopathies, and possibly high blood pressure (hypertension)), HIV infection, or an immunocomprised state.

Some embodiments described herein provide a combination of levocetirizine and montelukast for the prevention, modulation, and/or treatment of complications, signs, symptoms, and/or other effects associated with coronavirus disease.

In some embodiments, levocetirizine and montelukast as disclosed herein are used to treat the signs and/or symptoms caused by coronavirus disease. In some embodiments, levocetirizine and montelukast as disclosed herein are used to treat signs and/or symptoms originating from coronavirus disease.

In some embodiments, the one or more of the treated signs or symptoms of coronavirus disease are those not unique to coronavirus disease and/or can also be caused by other diseases that do not originate form coronavirus infection. In some embodiments, as disclosed herein, the combination of levocetirizine and montelukast is used specifically to treat signs or symptoms caused by coronavirus disease involving a coronavirus. In some embodiments, the combination of levocetirizine and montelukast as disclosed herein is not used to treat the signs and/or symptoms associated with non-coronavirus origins, even where those signs and/or symptoms overlap with those associated with coronavirus disease. Some embodiments, for example, include a step of selecting a patient to be treated that is suffering from coronavirus disease or is at risk for coronavirus disease. In some embodiments, the method of treating coronavirus disease, one or more signs and/or symptoms and/or complications thereof, includes the administration of levocetirizine and montelukast in an effective amount to a patient in need of treatment.

Without being bound to a particular mechanism, levocetirizine and montelukast may interact with and/or interfere with various biological cascades, including affecting different cascades (e.g., those involving NF-κB and/or leukotriene activation, respectively) in parallel to achieve their therapeutic effect. In some embodiments, levocetirizine and montelukast interrupt or interfere with one or more cascades involving one or more of cortisol, IκB kinase, IκB, COX-2, C-Jun Fos, MAPKs phosphatase I, Jun N-terminal kinase, MAPKs, MAPK-interacting kinase, calcium kinase II, calcium/calmodulin dependent kinase II, cPLA2a, 5-LOX, and the like.

In some embodiments, the combination of levocetirizine and montelukast can interfere with and/or attenuate the innate immune response thereby treating coronavirus disease. In some embodiments, the combination of levocetirizine and montelukast can interfere with coronavirus disease pathways through one or more of toll-like receptors (TLRs, including but not limited to, one or more of TLR-1, TLR-2, TLR-3, TLR-4, TLR-5, TLR-6, TLR-7, TLR-8, TLR-9, and/or TLR-10), LPS-binding protein (LBP), the opsonic receptors (e.g., CD14), and/or monocytic intracellular proteins (e.g., NOD1, NOD2, etc.). As shown in FIG. 2, LPS (the major component of the outer membrane of gram-negative bacteria) can be sensed via an LPS-binding protein (LBP)-LPS complex. In some embodiments, the combination of levocetirizine and montelukast can interfere with any of the signaling pathways shown, including those involving toll-like receptor 4 (TLR4)-MD-2 complex, the macrophage scavenger receptor (MSR), CD11b/CD18, and ion channels. In some embodiments, the combination of levocetirizine and montelukast can interfere with intracellular signaling that relies on binding intracellular TLR domain, TIR (Toll/IL-1 receptor homology domain), IRAK (IL-1 receptor-associated kinase), MyD88 (myeloid differentiation protein 88), TIRAP (TIR domain containing adapter protein, Tollip (Toll-interacting protein). In some embodiments, the combination of levocetirizine and montelukast can interfere with MyD88-independent pathways including those involving TIRAP/Mal signals through an RNA-dependent protein kinase (PKR) and interferon regulatory factor (IRF)-3. Intracellular receptors called NOD proteins (nucleotide-binding oligomerization domain) may also be involved in the mechanism. NOD1 may participate as an APAF-1-like activator of Caspase-9 (an enzyme critical to the apoptotic pathway) and Nf-kB. In some embodiments, expression of NOD1 and NOD2 confer responsiveness to Gram-negative LPS.

In some embodiments, the combination of levocetirizine and montelukast can down regulate the production of interferon-γ (IFN-γ) from toxin-activated T cells. In some embodiments, the combination of levocetirizine and montelukast can interfere with the activation of the intracellular protein complex NF-κB (nuclear factor kappa B) which is in turn responsible for the reduction of I-CAM-1. I-CAM-1, a transmembrane protein, is viewed as the portal of entry of human rhinovirus into the cell. In some embodiments, the combination of levocetirizine and montelukast decreases eosinophil and neutrophil quantity and migration and/or inflammatory mediators/cytokines/adhesion molecules: IL-1b, TNF-α, NF-kB, IL-4, IL-6, IL-7, IL-8, IL-12, IL-15, IL-18, RANTES, GM-CSF, TLR-3, AP-1, ICAM-1, and V-CAM-1. TNF-α (a potent signaling protein produced by macrophages/monocytes during acute inflammation), regulates in part, one or more symptoms of coronavirus disease (e.g., fever and other bodily responses to infectious exposure). TNF-α and IL-1 are cytokines that mediate many of the immunopathological features of LPS-induced shock which are released during the first 30-90 minutes after exposure to LPS. These cytokines activate a second level of inflammatory cascades including cytokines, lipid mediators and reactive oxygen species, as well as upregulating cell adhesion molecules that result in the initiation of inflammatory cell migration into tissues.

Exemplary chemokines, cytokines, and biomarkers that may be involved in the treatment of coronavirus disease that, in some embodiments, are downregulated or upregulated through the administration of levocetirizine and montelukast include but are not limited to: Granulocyte macrophage colony stimulating factor (GM-CSF); GROα; Interferon α2 (IFNα2); IFNβ; IFNγ; IL-10; Interleukin 12p70 (IL-12p70); IL12p40; Interleukin 1α (IL-1α); IL-1β; IL-1 receptor antagonist (IL-1RA); IL-2; IL-4; IL-5; IL-6; IL-8; IFN-γ-inducible protein 10 (IP-10); Monocyte chemoattractant protein 1, -2, or -3 (MCP-1, MCP-2, MCP-3); Monocyte chemoattractant protein 3 (MCP-3); Macrophage colony stimulating factor (MCSF); MIP-α; MIP-1β; Soluble CD40 ligand (sCD40L); Soluble E-selectin (sE-selectin); Soluble Fas ligand (sFasL); Tumor necrosis factor α and β (TNF-α and/or TNF-β); Vascular endothelial growth factor A (VEGF-A); D-dimer; Tissue plasminogen activator (TPA); Plasminogen activator inhibitor-1 (PAI-1); Serum amyloid antigen (SAA); Regulated on activation, normal T-cell expressed and secreted (RANTES); sVCAM-1; Fibrinogen; Ferritin; Cortisol; Tissue factor (TF); Thrombomodulin; S100B protein; Cellular prion protein (PrP^{C}); Ubiquitin C-terminal hydrolase-L1 (UCH-L1); choline (cell membrane damage); Myo-inositol (cell membrane damage or reactive astrogliosis); Tau protein; p-Tau (phosphorylated Tau); ICAM-1 (Intercellular adhesion molecule 1); ICAM-5 (Intercellular adhesion molecule 5); GFAP (Glial fibrillary acidic protein); NRGN (Neurogranin); SNCB (Beta-Synuclein); MT3 (Metallothionein 3); and injury specific exosomes/microRNA and NF-kB.

In some embodiments, the combination of levocetirizine and montelukast modulates, treats, or prevents threshold effects of coronavirus disease. In some embodiments, the combination of levocetirizine and montelukast modulates, treats, or prevents one or more of the signs, symptoms, and secondary conditions associated with coronavirus disease. For instance, in some embodiments, the combination of levocetirizine and montelukast modulates, treats, or prevents one or more of pain, nausea, vomiting, cramps, diarrhea, dehydration, electrolyte imbalance, fever, nervousness, confusion, headache, seizures, loss of consciousness, coma, altered cell signaling/trafficking, alterations in cellular differentiation and function, damage to immune/metabolic pathways, and vascular injury. In some embodiments, the combination of levocetirizine and montelukast reduces the risk and/or lessens the likelihood of a patient dying from coronavirus disease.

In some embodiments, the combination of levocetirizine and montelukast modulates, treats, or prevents coronavirus disease damage, signs, symptoms, and/or associated secondary conditions wherein the damage, sign, symptoms, and/or associated secondary conditions are inflammation-mediated. In some embodiments, the combination of levocetirizine and montelukast modulates, treats, or prevents coronavirus disease, signs, symptoms, and/or associated secondary conditions that are not IgE mediated. In some embodiments, the combination of levocetirizine and montelukast modulates, treats, or prevents inflammation-caused damage, signs, symptoms, and/or associated secondary conditions wherein the inflammation is caused specifically by coronavirus disease (and not other sources of inflammation). In some embodiments, the combination of levocetirizine and montelukast modulates, treats, or prevents one or more of damage, signs, symptoms, and/or associated secondary conditions associated with coronavirus disease that are not symptoms of allergy, cold, flu, sepsis, or radiation exposure.

Levocetirizine is an antihistamine and montelukast is a leukotriene receptor antagonist. Levocetirizine, as a potent H1-antihistamine, acts in part by down-regulating the H1 receptor on the surface of mast cells and basophils to block the IgE-mediated release of histamine—the agent responsible for the cardinal symptoms of the innate immune response, including an inflammatory response, fever, sneezing, rhinorrhea, nasal congestion, itchy palate, and itchy red and watery eyes. Levocetirizine offers a short time to peak plasma level, 0.9 hr, a short time to steady state level, 40 hours, a low volume of distribution, 0.4 L/kg, and an enhanced receptor affinity of 5×over first generation mepyramine in an acidic pH (many acute inflammatory disease states are associated with acidosis, a low physiologic pH; increased 5×). Levocetirizine has a 24-hour receptor occupancy of −75%, the highest of the commercially available antihistamines. Receptor occupancy of the second generation antihistamines appears to correlate with the pharmacodynamic activity in skin wheal and flare studies and with efficacy in allergen challenge chamber studies. Levocetirizine is approved in the US for the treatment of perennial allergic rhinitis and chronic idiopathic urticaria down to six months of age. Levocetirizine is the most potent of the five modern generation antihistamines through histamine induced wheal and flare data. For example, levocetirizine at 5 mg per day is more effective than fexofenadine at its commonly prescribed dose of 180 mg per day in the United States. In Europe the adult dose of fexofenadine is 120 mg per day.

Montelukast, a leukotriene receptor antagonist, acts by binding with high affinity and selectivity to the CysLT1 receptor to inhibit the physiologic actions of the leukotriene LTD4. Leukotrienes are fatty signaling molecules whose effects include airway edema, smooth muscle contraction and altered cellular activity associated with the inflammatory process. Overproduction of leukotriene is a major cause of inflammation. The cysteinyl leukotrienes (LTC4, LTD4, LDE4) are products of arachidonic acid metabolism. These leukotrienes are released from various cells including mast cells and eosinophils. They bind to receptors in the human airway and on other pro-inflammatory cells including eosinophils and certain myeloid stem cells. Without being bound to any particular theory, it is thought that overproduction of leukotrienes contributes to inflammation associated with coronavirus disease.

Montelukast is FDA approved in the US for the treatment of perennial allergic rhinitis, asthma, seasonal allergic rhinitis, and exercised induced bronchospasm. Montelukast is ineffective in improving asthma control or cold symptom scores caused by experimental rhinovirus infection. Analysis of secondary outcomes suggests that montelukast may protect against reductions in lung function and increases in sputum eosinophils caused by infections. During the recovery phase the percentage of sputum eosinophils was elevated in the placebo group, while the montelukast group remained at baseline levels. Further, peak expiratory flow was not decreased in the montelukast-treated patients. Montelukast treatment has no effect on the respiratory symptoms of patients with acute respiratory syncitial virus bronchiolitis.

Montelukast reaches a steady state level, like the second generation antihistamine, levocetirizine, in less than two days. Unlike other currently available leukotriene modulators, zileuton and zafirlukast, routine monitoring of liver function tests is not required. There are no drug interactions with warfarin, theophylline, digoxin, terfenadine, oral contraceptives, or prednisone.

Levocetirizine and montelukast are associated with millions of days of patient use; FDA approved in the United States for allergic disorders down to age six months. The combination of levocetirizine and montelukast can be given primarily or in conjunction with many of the existing therapeutic protocols for the treatment of complications associated with coronavirus disease. In some embodiments, the combination of levocetirizine and montelukast can be administered for the treatment of coronavirus disease or preventatively in patients, including pregnant women (both Pregnancy Category B) and children, that are under the age of about 1, about 2, about 3, about 4, about 5, about 10, about 15, or about 18. Moreover, both drugs have only once daily dosing, and no routine monitoring of blood work is necessary for most clinical situations. Further, both drugs exhibit minimal clinically relevant interactions with other medications. As described herein, both levocetirizine and montelukast reach steady state levels within two days to rapidly produce a synergistic and complementary anti-inflammatory effect.

Levocetirizine and montelukast are in different drug classes and target different receptors in the body. As disclosed elsewhere herein, they target different receptors in the body; levocetirizine and montelukast achieve their effect via different molecular pathways. In some embodiments, the combination of montelukast and levocetirizine achieves a unique synergy to treat and/or provide a protective effect against coronavirus disease, either prior to, during, or following bacterial, fungal, and/or viral exposure. In some embodiments, the synergistic effect shortens the course of complications caused by coronavirus disease and issues precipitated by coronavirus disease. In some embodiments, this synergistic effect is accomplished by the combination of levocetirizine and montelukast by targeting their respective different pathways in the body. In some embodiments, multiple inflammatory signaling pathways in the body are targeted to achieve protective effects or the treatment of coronavirus disease-based complications using levocetirizine and montelukast. In some embodiments, synergy is achieved by downregulating certain inflammatory processes. In some embodiments, the combination's effect to alleviate one or more disease states or symptoms associated with coronavirus disease exposure is achieved by stabilizing or reducing oxidative stress or physiological effects of oxidative stress caused by coronavirus disease. In some embodiments, synergy is achieved by enhancing certain antioxidant effects of the combination. In some embodiments, the use of the combination of montelukast and levocetirizine decreases one or more of the symptoms of, the duration of, morbidity from, and mortality from coronavirus disease-related disease states and symptoms. In some embodiments, the combination of levocetirizine and montelukast decreases the progression of complications associated with coronavirus disease. In some embodiments, the combined levocetirizine and montelukast therapy can improve quality of life by ameliorating one or more of the symptoms, side effects, and the underlying coronavirus disease damage or complication itself, resulting in decreased health-care costs. In some embodiments, a synergistic effect can be observed in the use of a combination of levocetirizine and montelukast to treat inflammation.

In some embodiments, levocetirizine and montelukast can be used in combination with other treatments for coronavirus disease, including one or more antibiotics, vasopressors, corticosteroids, insulin, immune stimulants, painkillers, and sedatives. In some embodiments, levocetirizine and montelukast can be used in combination with broad spectrum antibiotics including one or more of tetracycline, ciprofloxacin, levofloxacin, penicillin, cephalexin, meropenem, imipenem, piperacillin-tazobactam, tigecycline, metronidazole, aztreonam, cefepime, azithromycin, vancomycin, ceftriaxone, clindamycin, trimethoprim/sulfamethoxazole, doxycycline, linezolid, gentamycin, amikacin, tobramycin, or combinations thereof.

Without being bound to any particular theory, it is believed that unchecked, pro-inflammatory reactions in the body can exacerbate biological effects and issues caused by coronavirus disease. In some instances, these inflammatory responses contribute to the development and progression of complications associated with coronavirus disease exposure. In other instances, these inflammatory responses are themselves responsible for certain symptoms related to coronavirus disease. In some embodiments, levocetirizine and montelukast act by down regulating pro-inflammatory mediators elicited by evolving coronavirus disease, allowing the body to more readily react and recover from coronavirus disease and complications associated with coronavirus disease. In some embodiments, the levocetirizine and montelukast directly improve and/or resolve issues, signs, or symptoms caused by coronavirus disease. Some embodiments provide the combination of levocetirizine and montelukast as a medicament for the treatment of complications associated with coronavirus disease that are exacerbated by or result from innate immune responses or adaptive immune responses caused by coronavirus disease.

Without being bound to any particular theory, the anti-inflammatory effect of the combination of levocetirizine and montelukast is due, at least in part, to the fact that both levocetirizine and montelukast affect eosinophil migration/quantity; the eosinophil is considered by scientists/clinicians as one hallmark of inflammation. Additionally, as discussed elsewhere herein, the response may be related, at least in part, due to levocetirizine's interference with the toll-like receptors (TLRs) and montelukast's separate interference with the leukotriene-related pathways to inflammation.

A common feature of all TLR recognition is the activation of three major signaling pathways: nuclear factor kappa-B (NF-κB), mitogen-activated protein kinase (MAPKs), and one or more of the interferon regulatory factors (IRFs). In some embodiments, the combination of levocetirizine and montelukast is used in methods to treat complications associated with coronavirus disease by blocking activation of one or more of these pathways. NF-κB plays a pivotal role across a spectrum of inflammation, immunity, cell proliferation, differentiation, cell survival, and cell death. NF-κB is expressed in almost all cell types and tissues. Specific binding sites are present in the promoters/enhancers of a large number of genes. For example, NF-κB target genes include: Cytokines/Chemokines and their Modulators, Immunoreceptors, Proteins Involved in Antigen Presentation, Cell Adhesion Molecules, Acute Phase Proteins, Stress Response Genes, Cell Surface Receptors, Regulators of Apoptosis, Growth Factors, Ligands and their Modulators, Early Response Genes, Transcription Factors and Regulators, Viruses, and Enzymes.

In some embodiments, the combination of levocetirizine and montelukast is used in methods to treat complications associated with coronavirus disease that elicit cellular activity or inflammatory responses via NF-κB. In some embodiments, the combination of levocetirizine and montelukast treats complications associated with coronavirus disease by blocking activation through the NF-κB pathway. In some embodiments, the combination of levocetirizine and montelukast treats complications associated with coronavirus disease by blocking TLR activation through the NF-κB pathway and at least one other cellular signaling pathway selected from the group consisting of the MAPKs pathway and the IRFs pathway. In some embodiments, the combination of levocetirizine and montelukast treats complications associated with coronavirus disease by blocking cellular signaling pathways other than those mediated by TLRs. In some embodiments, the combination of levocetirizine and montelukast reduces the activation of the NF-κB/toll-like receptors and/or other intracellular or extracellular protein complexes (e.g., exosomes, histones). In some embodiments, the combination of levocetirizine and montelukast treats complications associated with coronavirus disease that are activated at least in part through NF-κB.

One example of the influential nature the NF-κB family of transcription factors is RANTES (regulated on activation, normal T cell expressed and secreted). In the ‘late’ or adaptive phase of the immune response, RANTES is a chemokine generally expressed three to five days after T-cell activation. RANTES expression, mediated exclusively through NF-κB, attracts eosinophils, monocytes, mast cells and lymphocytes, activates basophils and induces histamine release from these cells. Select H1 receptor antagonists (e.g., levocetirizine) have the remarkable ability to inhibit NF-κB and activator protein-1 (AP-1) activity though H1 receptor-dependent and independent mechanisms.

Levocetirizine has been shown to inhibit human rhinovirus (HRV)-induced ICAM-1, cytokine expression, and viral replication in airway epithelial cells from both the nose and lung. Overexpression of the H1 receptor in the laboratory resulted in the inhibition of the HRV-induced upregulation of ICAM-1, 11-6, TLR3 expression and NF-κB activation. Levocetirizine reduced the levels of HRV-induced increases in ICAM-1 regardless of whether the levocetirizine was added before, after, or at the time of the HRV infection. The results were in agreement with previous research on the inhibitory effects of levocetirizine ICAM-1 up-regulation.

In some embodiments, the methods described herein involve identifying a patient (e.g., a subject) in need of treatment. In some embodiments, a patient may comprise any type of mammal (e.g., a mammal such as a human, cow, sheep, horse, cat, dog, goat, rodent, etc.). In some embodiments, patients in need of treatment include those who are at risk for contracting coronavirus disease, have had their circulatory system compromised (e.g., by a cut, through surgery, wounds), etc.), or that are suffering from coronavirus disease. In some embodiments, patients at risk for coronavirus disease include subjects having undergone surgery, patients in the ICU, patients with an in-dwelling catheter, the elderly (e.g., equal to or greater than 65, 75, or 80 years of age), the young (e.g., equal to or less than under 5, 7, or 10 years of age), immunocompromised patients (e.g., those undergoing chemotherapy treatment, those on mechanical ventilation, those suffering from HIV, leukemia, cancer, pneumonia, lung infection, kidney infection, infections of the abdominal region, etc.). In some embodiments, patients in need of treatment can include those who are at a high likelihood of developing complications associated with coronavirus disease due to lifestyle variables (e.g., working in an area where microbes or viruses are present, etc.). In some embodiments, for patient groups, the combination of levocetirizine and montelukast can be administered preventatively for at risk patients or curatively patients suffering from coronavirus disease. In some embodiments, for patient groups (e.g., at risk or suffering from coronavirus disease), the combination of levocetirizine and montelukast can be administered preventatively or curatively after about age: 1, 5, 10, 20, 30, 40, 50, 60, 70, 80, 90, or ranges including and/or spanning the aforementioned values, and throughout the rest of the patient's life.

Once identified as a patient, the combination of levocetirizine and montelukast is administered to the patient for a period of time. In some embodiments, the period of administration comprises a period starting when the patient first displays symptoms, or when the patient has displayed symptoms for a period of more than about 1 hour, 1 day, about 2 days, or ranges spanning and/or including the aforementioned values. In some embodiments, the combination is administered until a time when the complications associated with coronavirus disease are controlled or cured (e.g., the acute symptoms have subsided, symptoms have decreased to a baseline, risk factors for death have decreased, etc.), or for a prescribed period of time of less than about 1 week, about 2 weeks, about 3 weeks, about a month, about two months, about 6 months, or about a year. In some embodiments, the period of time comprises a period spanning from when the patient or an administrator of treatment (e.g., a doctor, nurse, medic, technician, relative, etc.) suspects the patient has coronavirus disease to a time when the patient is no longer at risk of developing complications associated with coronavirus disease. In some embodiments, the combination of levocetirizine and montelukast is given to alleviate symptoms of coronavirus disease and the combination is given for the duration of the symptoms. In some embodiments, the combination of levocetirizine and montelukast is administered preventatively for a period during high exposure risk or during a period when the coronavirus disease exposure is likely (e.g., working in remote areas, where coronavirus disease is common, etc.).

In some embodiments, dosing and delivery of the combination of levocetirizine and montelukast can be performed for periods between five days-twelve months to achieve continuous tissue levels of the drug combination. In some embodiments, dosing and delivery of levocetirizine and montelukast can be performed for periods of at least about: 1 day, 5 days, 10 days, 20 days, 30 days, 50 days, 100 days, 200 days, 300 days, or ranges including and/or spanning the aforementioned values. In some embodiments, the rationale is to achieve sustained tissue levels to modulate NF-κB at multiple targets within the immune system (Constant overexpression of the H1 Receptor).

In several embodiments, using the combination as disclosed herein, the average time course of coronavirus disease (or a symptom or indicator thereof) is shortened by equal to or at least about: 10%, 20%, 30%, 40%, 50%, 60%, 70%, or ranges including and/or spanning the aforementioned values. In several embodiments, using the combination as disclosed herein, the average time course of coronavirus disease or a symptom or indicator thereof is shortened by equal to or at least about: 2.5 days, 5 days, 7.5 days, 10 days, or ranges including and/or spanning the aforementioned values. In several embodiments, using the combination as disclosed herein, the average time course of coronavirus disease is reduced to a period of equal to or at least about: 2.5 days, 5 days, 7.5 days, 9 days, or ranges including and/or spanning the aforementioned values.

In some embodiments, the levocetirizine montelukast combination is administered in a sequential manner. In some embodiments, levocetirizine is administered first. In some embodiments, montelukast is administered first. In some embodiments, the combination is administered in a substantially simultaneous manner.

In some embodiments, the combination is administered to the patient by one or more of the routes consisting of enteral, intravenous (including, but not limited to a long-acting injectable, e.g., an extended-release preparation), intraperitoneal, inhalation, intramuscular (including, but not limited to a long-acting injectable, subcutaneous and oral). In some embodiments, the levocetirizine and montelukast are administered by the same route. In some embodiments, the levocetirizine and montelukast are administered by different routes. In some embodiments, the combination is dosed to the patient using an effective amount of a combination of levocetirizine and montelukast.

In some embodiments, levocetirizine and montelukast are provided in long-acting delivery formats to treat the complications associated with coronavirus disease (or for a prophylactic period). In some embodiments, the long-acting delivery formats deliver therapeutic doses of levocetirizine and montelukast for periods of at least about: 1 week, 2 weeks, 1 month, 6 months, or ranges including and/or spanning the aforementioned values. In some embodiments, levocetirizine and montelukast are provided in fast-acting delivery formats to treat the complications associated with coronavirus disease. In some embodiments, the levocetirizine and montelukast are provided in once-daily or multiple-daily doses. In some embodiments, traditional oral delivery systems: film strips, bilayer tablets, capsules, tablets, nebulized therapy, etc. could be utilized if administered on at least a twice daily regimen, early in the course of the complication, i.e., the first seventy-two hours. Otherwise, with the onset of nausea and diarrhea, or manifestation of any other systemic indicator, e.g., shortness of breath, hypotension, rapid pulse, fever, etc., an IV (intravenous), IM (intramuscular) or LAI (long-acting injectable) can be successful in changing the outcome.

Depending upon the patient's age, weight, BMI (body mass index) and severity of the disease on presentation, the dosing (oral, IV, IM) or dose (LAI) can be titrated to effect over the following range:

Levocetirizine: 1.25 mg-30 mg/24 hours

Montelukast: 4 mg-50 mg/24 hours for a duration of at least five days

In some embodiments, the dose is adjusted depending on the patient's response to the combination or depending on the progression of the disease state.

In some embodiments, the typical daily dosage for levocetirizine is about 5 mg, about 10 mg, about 15 mg for adults. Studies in humans have shown that doses of levocetirizine up to 30 mg/day can be safely administered. In some embodiments, daily doses of levocetirizine can be at least about 1 mg, about 5 mg, about 10 mg, about 20 mg, about 30 mg, about 40 mg, about 50 mg, about 100 mg, about 500 mg, or ranges including and/or spanning the aforementioned values. Montelukast, a leukotriene receptor antagonist, acts concurrently to protect the airway as well as to block mediators in the inflammatory cascade. The typical daily dosage of montelukast is 10 mg for adults. Montelukast has been administered at doses up to 200 mg/day to adult patients for 22 weeks and in short-term studies, and up to 900 mg/day to patients for approximately one week without clinically relevant side effects. In some embodiments, daily doses of montelukast can be at least about 1 mg, about 5 mg, about 10 mg, about 20 mg, about 30 mg, about 40 mg, about 50 mg, about 100 mg, about 200 mg, about 400 mg, about 600 mg, about 800 mg, about 1000 mg, about 2000 mg, about 4000 mg, about 6000 mg, or ranges including and/or spanning the aforementioned values.

In some embodiments, levels of levocetirizine utilized in the laboratory model can be safely achieved in a clinical setting; however, are above the standard adult dose of 5 mg daily used for the treatment of allergy and asthma. In some embodiments, the addition of montelukast, also above the standard 10 mg adult dose for allergy and asthma results in a remarkable synergistic effect which has been shown in our clinical setting to safely decrease the symptoms and duration of select viral infections (e.g., human rhinovirus, influenza).

Given the half-lives of the molecules and other pharmacokinetic considerations, once oral daily dosing, particularly in acutely ill patients, may not be effective. As such, in some embodiments, in a difficult-to-treat or harsh environment, a long-acting injectable may be employed. In some embodiments, a formulation (e.g., a long-acting injectable) comprising 50-100 mg of levocetirizine and 100-200 mg of montelukast within a pharmaceutically acceptable medium or as a pharmaceutically acceptable medium (e.g., reconstituted lyophilized powder) is dosed to maintain a steady state level for seven days. In some embodiments, the injectable can be configured to deliver the oral equivalent of between 5 mg and 20 mg of levocetirizine and between 10 mg and 40 mg of montelukast to the patient per day (depending on the nature and extent of the disease process; taking into consideration patient weight, age, etc.). In some embodiments, oral dosing can also be used where appropriate dosing is between 5 mg and 20 mg of levocetirizine and between 10 mg and 40 mg of montelukast/day, respectively. Divided oral daily dosing may be employed. In some embodiments, the formulation comprises about 50 mg, about 100 mg, about 150 mg, about 200 mg, about 300 mg, about 400 mg, about 500 mg, or more of levocetirizine. In some embodiments, the formulation comprises about 50 mg, about 100 mg, about 150 mg, about 200 mg, about 300 mg, about 400 mg, about 500 mg, or more of montelukast.

In some embodiments, long-acting comprises injectables that peak in a short period of time (e.g., within about 1-3 hours, or less than about 1 hour, about 2 hours, about 3 hours, about 4 hours, about 5 hours, or ranges including and/or spanning the aforementioned values). In some embodiments, long-acting injectables are those that maintain a nearly constant plasma or CNS level for a sustained period of time (e.g., at least about 3 days, about 4 days, about 5 days, about 6 days, about 7 days, about 14 days, about 21 days, about 28 days or more, or ranges including and/or spanning the aforementioned values). In some embodiments, a nearly constant blood concentration is one that is about 25 ng/mL (combined plateau of both drugs), about 50 ng/mL, about 150 ng/mL, about 250 ng/mL, about 350 ng/mL, about 450 ng/mL, about 550 ng/mL, about 650 ng/mL more than about 650 ng/mL, or ranges including and/or spanning the aforementioned values (plus or minus about 25-50 ng/mL).

The technology has evolved to repurpose levocetirizine+montelukast in a long-acting injectable. This concept is particularly useful: (a) where the patient is unable to swallow, (b) where the patient is unconscious, (c) where there are limited resources for overall care and management, (d) for prophylaxis in a time of war, (e) for use as a bioterrorist counteragent, and (f) during travel in space.

Predictive modelling software can be utilized to take existing information on the API (active pharmaceutical ingredient), excipients, the desired release profile, and end environment (body v CNS) and calculate a formulation which can then be used to manufacture microparticles that encapsulate the API and release it at a desired rate. Using computer metrics, the laboratory to manufacturing formulation variances can be minimized during the design phase.

Delivery vehicles include but are not limited to injectable microparticles, nanoparticles, matrix implants, and device coatings. Release profiles can be designed as constant rate (where doses are released at desired profiles for a period of days, weeks, or months), delayed release, or sequential release. In some embodiments, a wide variety of controlled release systems can be formulated. In some embodiments, the delivery vehicle is selected from the group consisting of injectable microparticle, nanoparticles, pellets, rods discs, tablets, thin film coatings, matrix implants, device coatings, and combinations thereof. In some embodiments, the delivery vehicle formulated from one or more of Poly(lactic-co-glycolic acid) (PLGA), Polyanhydrides (PSA, PSA:FAD), Polylactides (PLA), Poly-ortho-esters (POE), or HPMC hydrogels. The release profile can be tailored between Constant Rate (days, weeks, months), Delayed Release, and Sequential Release.

Without being bound to a particular theory, delivery of levocetirizine and montelukast (e.g., sustained, intermittent, or otherwise) will stabilize NF-κB through the overexpression of the H1-receptor in a dose-dependent manner.

In some embodiments, oral BID dosing can be used to saturate levocetirizine and montelukast receptors in an estimated ratio of 3 mg/6 mg (respectively) one in the AM and two HS. Separately, 6 mg/12 mg at night for long-term treatment. In some embodiments, where therapy would be long-term, months to years, qd to bid with an optimal daily dosing range of 6-9 mg/12-18 mg: levo/monte; titrated to effect as determined from monthly to quarterly patient visits, neuropsychiatric assessments at six month intervals and QOL questionnaires at each patient visit. In some embodiments, both molecules cross the blood-brain barrier at 0.1 mg/kg. In some embodiments, lower (or higher) dosing could be used.

In some embodiments, the combination of levocetirizine and montelukast can be given instead of, or in conjunction with, existing therapeutic protocols for the treatment of coronavirus disease.

In some embodiments, the combination of levocetirizine and montelukast (or one or more of levocetirizine and montelukast) is formulated for intravenous (IV) delivery. In some embodiments, one or more of levocetirizine and montelukast, or the combination of levocetirizine and montelukast is formulated in combination with one or more intravenous antibiotics. In some embodiments, one of levocetirizine and montelukast is administered intravenously while the other is administered orally or by another route as disclosed herein. In some embodiments, one or more of levocetirizine and montelukast is administered intravenously while an antibiotic is administered orally or by another route as disclosed herein. In some embodiments, one or more of levocetirizine and montelukast is administered orally while an antibiotic is administered intravenously or by another route as disclosed herein.

Some embodiments include a kit comprising the combination of levocetirizine and montelukast. In some embodiments, the kit includes a saline IV bag. In some embodiments, the kit includes instructions for mixing one or more of the combination of levocetirizine and montelukast with the IV saline. In some embodiments, the kit includes one or more of needles, tubing, syringes, antiseptic swabs, or the like.

### EXAMPLES

Levocetirizine, a third-generation antihistamine, and montelukast, a leukotriene receptor antagonist, exhibit remarkable synergistic anti-inflammatory activity across a spectrum of signaling proteins, cell adhesion molecules, and leukocytes. By targeting cellular protein activity, they are uniquely positioned to treat the symptoms of COVID-19. Clinical data to date with an associated six-month follow-up, suggests the combination therapy may prevent the progression of the disease from mild to moderate to severe, as well as prevent/treat many of the aspects of ‘Long COVID,’ thereby cost effectively reducing both morbidity and mortality.

To investigate patient outcomes, 53 consecutive COVID-19 test (+) cases (ages 3-90) from a well-established, single-center practice in Boston, Mass., between March and November 2020, were treated with levocetirizine and montelukast in addition to then existing protocols. The data set was retrospectively reviewed. Thirty-four cases were considered mild (64%), 17 moderate (32%), and 2 (4%) severe. Several patients presented with significant comorbidities (obesity: n=22, 41%; diabetes: n=10, 19%; hypertension: n=24, 45%). Among the cohort there were no exclusions, no intubations, and no deaths. The pilot study in Massachusetts encompassed the first COVID-19 wave which peaked on Apr. 23, 2020 as well as the ascending portion of the second wave in the fall. During this period the average weekly COVID-19 case mortality rate (confirmed deaths/confirmed cases) varied considerably between 1-7.5%. FDA has approved a multicenter, randomized, placebo-controlled, Phase 2 clinical trial design, replete with electronic diaries and laboratory metrics to explore scientific questions not addressed herein.

### Example 1: Method

All patients were screened for psychological conditions using the Patient Health Questionnaire-4 (PHQ-4). Patients testing (+) for COVD-19 within the clinical practice or hospital and subsequently referred to the investigator by another provider, were sequentially seen and treated with the combination of levocetirizine and montelukast. All patients were accepted for treatment regardless of presenting symptoms; no patients were excluded due to underlying comorbidities. Follow-up consisted of a minimum six-month period.

Among the patient population were 32 females and 21 males. The mean age among males was 55 and females, 51. Fifteen patients (28%) were between the ages of 66 and 90; 11 patients (21%) were under 30. Thirty-four cases were considered mild (64%), 17 moderate (32%), and 2 (4%) severe.

Moderate was defined as shortness of breath (difficulty breathing) with or without any of the other symptom of mild COVID-19. Clinical signs suggestive of moderate illness with COVID-19 were defined as a respiratory rate≥20 breaths per minute, saturation of oxygen (SpO2)>93% on room air at sea level, and heart rate≥90 beats per minute. In the 18 hospitalized patients (34%), therapy was initiated upon diagnosis. The 2 severe cases received remdesivir as well as levocetirizine and montelukast, the latter of which were initiated on hospital day 9. With the exception of one patient with nasal polyps, steroids were not part of the treatment paradigm. In addition, no patient received monoclonal antibodies. Within the combined outpatient and inpatient cohort, 22 were considered obese (BMI>30, 41%), 10 had diabetes (19%) and 24 had hypertension (45%).

### Example 2: Formulation and Dosing

The current study utilized commercially available products and the respective adult doses for the treatment of allergy and asthma, i.e., levocetirizine 5 mg and montelukast 10 mg orally, once a day. In general, therapy was continued for 14 days. The three-year-old pediatric patient was treated with levocetirizine 1.25 mg and montelukast 4 mg daily, also for 14 days. Patients with significant comorbidity were treated for thirty days or longer, depending upon their underlying diagnoses (e.g., asthma, allergy, nasal polyps, etc.). Clinical experience with the treatment of COVID-19 outside the pilot study as well as treatment of multiple other inflammatory disease states (e.g., sepsis, traumatic brain injury, traumatic lung injury, vasculitis) over the past 10 years, suggests a potentially higher, yet safe dosing regimen may foreshorten the nature and extent of the COVID-19 presentation, particularly if therapy is initiated early (within 5 days of the onset of symptoms/diagnosis). Such patients are less likely to progress to pneumonia or require hospitalization, parameters which have been defined in the Phase 2 trial design. Key characteristics of levocetirizine and montelukast are summarized in Table 1 below.

TABLE 1

| Levocetirizine | Montelukast |
|---|---|
| A leading H1 receptor antagonist in the | A leading leukotriene modulator in the |
| world among more than 40 | world |
| antihistamines | FDA approved for allergy, asthma, and |
| Considered an ideal, H1 receptor | exercise induced bronchospasm |
| antagonist, ‘insurmountable’ by | Pregnancy Category B |
| pharmacologists with a Vd 0.4 L/kg; | Titratable from 4 to 40 mg with linear |
| ideal molecule Vd <0.6 L/kg | pharmacokinetics to 50 mg/day |
| FDA approved for allergic rhinitis, | Safety studies at 200 mg/day for 22 |
| chronic idiopathic urticaria (CIU)41 | weeks; 900 mg/day for approximately |
| Pregnancy Category B | one week |
| Titratable with increasing efficacy | Ideal in COVID-19 acute care medicine |
| demonstrated in CIU from 5 to 20 | where the lung is the target organ |
| mg/day | Given orally/nasogastric tube improves |
| Only antihistamine in the world to | FEV1: 15% in one to three hours |
| independently improve quality of life | Efficiently attenuates ARDS in a mouse |
| across all domains (global health status | model |
| SF-36; P < 0.001 for all scales) as well as | Antiviral activity (disrupting viral |
| decrease overall health-care costs in a | integrity) against Zika virus, Dengue |
| series of 421 patients with allergy/asthma | virus, and yellow fever virus (like |
| treated for six months39 | COVID-19 and Human rhinovirus |
| More potent and safer than astemizole, | (HRV), all are ssRNA viruses) |
| the latter, a second-generation | Potential dual COVID-19 activity main |
| antihistamine with antiviral activity); | protease enzyme inhibition and virus |
| astemizole was active against both | entry into the host cell (Spike/ACE2) |
| SARS-CoV and MERSCoV. Astemizole; | |
| however, was withdrawn from the US | |
| market in 1999 due to cardiac toxicity - | |
| prolongation of the QTc interval | |
| Cell and clinical science - antiviral | |
| activity against human rhinovirus-16 | |
| (HRV-16) | |

Levocetirizine, a third-generation antihistamine, classically downregulates the H1 receptor on the surface of mast cells and basophils to block the IgE-mediated release of histamine. Histamine has been well characterized by its effects on the body, including in part, its function as a neurotransmitter, dilation of blood vessels which in turn increases permeability and lowers blood pressure, contraction of smooth muscle in the lung, uterus, and stomach, and as a source of sneezing, itching, and congestion. Levocetirizine is considered by pharmacologists an ‘insurmountable’ H1 receptor antagonist. It has been objectively established as the most potent of the five modern generation antihistamines (levocetirizine, cetirizine, fexofenadine, loratadine, and desloratadine) through histamine wheal and flare data.

Levocetirizine, given its low volume of distribution and high receptor occupancy, is also among a select group of H1 receptor antagonists which can inhibit NF-kB and activator protein-1 (AP-1) activity through H1 receptor-dependent and independent mechanisms_{9,21,22}. Induction of such activity follows in a dose-dependent manner to decrease, inter alia, tumor necrosis factor-α induced production of the chemokine RANTES (Regulated upon activation, normal T cell expressed and presumably secreted). RANTES expression, mediated exclusively through NF-kB, attracts eosinophils, monocytes, mast cells and lymphocytes, activates basophils, and induces histamine release from these cells.

Montelukast functions at the CysLT1 receptor to inhibit the physiologic action of leukotriene D4 (LTD4). Leukotrienes are protein mediators of inflammation similar to histamine; however, 100-1000×more potent on a molar basis than histamine in the lung. LTD4 is the most potent cysteinyl leukotriene in contracting smooth muscle, thereby producing bronchoconstriction. Contemporary cell and animal science support the use of montelukast in patients with acute respiratory distress syndrome.

At the molecular level, distinct from CysLTR1 antagonism, montelukast has also been reported to inhibit the activation of NF-kB in a variety of cell types including monocytes/macrophages, T cells, epithelial cells, and endothelial cells, thereby interfering with the generation of multiple proinflammatory proteins. Separately, Robinson, et al. found that montelukast independently inhibited resting and GM-CSF-stimulated eosinophil adhesion to VCAM-1 under flow conditions.

An expanding body of molecular science favorably supports montelukast as a potential therapeutic for the treatment of COVID-19. Multiple in silico and in vitro studies have depicted the dual potential of montelukast to inhibit the SAR-CoV-2 main proteinase 3CL^{pro} as well as viral entry into the host cell (Spike/ACE2). The anti-inflammatory drugs montelukast, ebastine, a second-generation antihistamine, and steroid, Solu-Medrol (methylprednisolone) exhibit remarkable affinities to 3CL^{pro}. 3CL^{pro} plays an essential role in processing polyproteins, the resultant products of which are subsequently utilized in the production of new virions. Additionally, there is a known clinical crossover between ebastine and levocetirizine, the latter considered more potent.

Montelukast has been safely and extensively used throughout the world since 1998. In certain patient populations, particularly children, are reports of an increase incidence of neuropsychiatric events (NAE). As such, FDA issued a black box warning in the Spring of 2020 pertaining to use in allergic rhinitis. However, observational studies, including the FDA's own Sentinel study which examined asthma patients 6 years and older, found no increased risk of mental health side effects with montelukast compared to inhaled corticosteroids (ICS). Moreover, in those with a psychiatric history, montelukast patients exhibited a decreased risk of outpatient depression compared to ICS patients; additional data found no statistical association (inpatient depressive disorder and self-harm) between montelukast and serious NAEs, across age, sex, and time strata₃₅. The absence of adverse outcomes was consistent with results from clinical trials and well-conducted observational studies. In their conclusion, from the totality of the observational evidence, including well-conducted observational studies, montelukast was not suggestive of a risk. Prudence; however, dictates that patients considered for therapy undergo a mental health screening. Levocetirizine has also been used extensively across the globe beginning with a successful launch in Europe at the turn of the century. It remains the only antihistamine in the world to demonstrate improved quality of life across all treatment domains (Short Form Health Survey-36 (SF-36); p<0.001) in a series of 421 patients with allergy/asthma treated for six months₃₉. The SF-36 addresses multiple domains: physical functioning, role limitation to due physical health, bodily pain, social functioning, general mental health, role limitation due to emotional problems, vitality/fatigue, and general health perception.

The two molecules are titratable, i.e., levocetirizine from 5 mg-20 mg/day and montelukast from 10 mg to 40 mg/day and are underscored by millions of days of patient use. In the United States, both are considered Pregnancy Category B (dosed once daily—levocetirizine 5 mg; montelukast 10 mg). In the context of treating a potentially life-threatening infectious disease, the combination appears remarkably suited as a therapeutic in the COVD-19 treatment paradigm.

In view of the anti-inflammatory synergy between levocetirizine, a third generation antihistamine, and montelukast, a leukotriene receptor antagonist, the combination is ideally positioned to treat COVID-19 symptoms, addressing multiple targets within the inflammatory pathway including: histamine, leukotriene D4 (LTD4), NF-kB, ICAM-1, VCAM-1, IL-4, IL-6, IL-8, RANTES, GM-CSF, TLR-3, AP-1, eosinophil and neutrophil quantity and migration.

The downregulation of NF-kB is considered a key mechanism of action (MOA) for relief of COVID-19 symptoms and mitigation of inflammation as NF-kB plays a critical role in mediating responses to a remarkable diversity of external stimuli; providing at least in part, regulation of cytokine release triggered by infection. Equally if not more important, is recognition of the NF-kB family of transcription factors as pivotal across the spectrum of not only inflammation, but also immunity, cell proliferation, differentiation, cell survival, and cell death. NF-kB is expressed in almost all cell types and tissues. Specific binding sites are present in the promoters and/or enhancers of a large number of genes including: cytokines/chemokines and their modulators, immunoreceptors, proteins involved in antigen presentation, cell adhesion molecules, acute phase proteins, stress response genes, cell surface receptors, regulators of apoptosis, growth factors, ligands and their modulators, early response genes, transcription factors and regulators, viruses, and enzymes.

### Example 3: Results

A descriptive analysis of 53 COVID-19 (+) patients from a well-established single-center otolaryngology and allergy practice is presented in Table 2. The pilot study in Massachusetts encompassed the first COVID-19 wave which peaked on Apr. 23, 2020 as well as the ascending portion of the second wave in the fall. During this time the average weekly COVID-19 case mortality rate (confirmed deaths/confirmed cases) varied considerably between 1-7.5%.

During the course of the illness 66% had a fever (n=35; >100.4° F., 38° C.), 51% had a headache (n=25) and 28% had loss of the sense of smell/taste (n=15). Fifty-one of 53 patients were considered a clinical cure on therapy with restoration of their overall status to a pre-infection baseline within 2 weeks. Two patients, ages 73 and 80, continued to complain of fatigue for a period of time post discontinuation of therapy. The 73-year-old male diagnosed in March 2020, improved in 10 days although continued to exhibit a dry cough for months. The 80-year male, post subdural hematoma with a neurological deficit, was diagnosed in the hospital on day 3; however, did well and also recovered from the virus on combination therapy.

Many allergy and asthma patients had co-existing morbidities including obesity, diabetes and hypertension, which increased their risk for major complications associated with COVID-19, yet notably recovered well from the virus. Early treatment, particularly in younger patients, enhanced the clinical response, with resolution of headache and fever within the first 48 hours following initiation of therapy. Analyzed collectively, the data support improved patient outcomes for those treated with the combination of levocetirizine and montelukast over patients who were either left untreated or treated with the then existing protocols.

Most patients treated with co-administration of levocetirizine and montelukast had symptom resolution within 7 days versus 10-14 days or longer reported by untreated symptomatic patients. Subjects with symptom resolution after 7 days typically had comorbidities that required a longer treatment period. Notably, there were no comorbidity exclusions, no intubations, no deaths, and no reported treatment-related safety findings. In addition, no one in the study exhibited ‘Long COVID’ symptoms greater than three months.

These data suggest the combination therapy, underscored by their uniquely synergistic mechanisms of action, contributes to symptom relief for patients testing positive for COVID-19. The data also suggest that the two drugs can be safely co-administered in COVID-19 patients over a wide age range (3-90), even those with significant comorbidities.

Limitations of the pilot study include the absence of a placebo arm, respectfully considered within the ethical constraints of the underlying disease. Regarding statistics, data was collected from March-November 2020, a period in time when there was insufficient testing, potentially inflating the treatment effect. Without controls, the extent of this effect is difficult to quantify. Further study is warranted.

Strengths of the pilot study include the mitigation of symptoms, particularly given the intrinsic mechanism of action of montelukast, inter alia, its ability to improve breathing. Moreover, treatment was offered to all patients regardless of age, comorbidities, and time from presentation of symptoms to time to the initiation of therapy. FDA accepted the initial data as positive proof of concept, suggested, and subsequently approved a multicenter, randomized, placebo-controlled, Phase 2 clinical trial design, replete with electronic diaries and laboratory metrics to explore scientific questions not addressed herein.

TABLE 2
Clinical Overview, Symptoms, and Comorbidities in 53 COVID-19 (+) Patients

| | | Clinical Overiew | | | | | | | | | |
|---|---|---|---|---|---|---|---|---|---|---|---|
| | | | Initial | Symptoms | | | | | | | |
| | | Outcome (cured, | severity of | | | | Loss of | | Comorbitities | | |
| | | still symptomatic | symptoms (mild | | Thoracic | | smell | | | | Hyper- |
| | | still very ill, | moderate | Cough | Tightness | Fever | taste | Headache | Obesity | Diabetes | tension |
| Sex | Age | deceased) | severe) | (Y/N) | (Y/N) | (Y/N) | (Y/N) | (Y/N) | (Y/N) | (Y/N) | (Y/N) |
| M | 54 | CURE | MOD | Y | Y | Y | N | N | Y | N | Y |
| M | 69 | CURE | MILD | Y | Y | Y | N | Y | N | N | N |
| M | 58 | CURE | MOD | Y | N | Y | N | N | N | N | Y |
| M | 63 | CURE | MOD | Y | Y | Y | N | | Y | N | Y |
| M | 62 | CURE | MOD | Y | N | Y | N | | Y | N | Y |
| F | 67 | CURE | MILD | N | N | Y | Y | N | N | N | N |
| F | 24 | CURE | MILD | Y | N | N | N | Y | N | N | N |
| F | 40 | CURE | MILD | N | N | N | N | Y | N | N | N |
| F | 56 | CURE | MILD | Y | Y | Y | N | Y | N | N | N |
| F | 73 | FATIGUE | MILD | Y | N | Y | Y | Y | N | N | N |
| M | 31 | CURE | MILD | N | N | N | Y | N | N | N | N |
| M | 44 | CURE | MOD | Y | N | N | N | N | N | N | N |
| M | 40 | PARTIAL | MOD | Y | Y | Y | Y | Y | Y | N | N |
| | | SMELL | | | | | | | | | |
| M | 61 | CURE | MILD | Y | N | Y | Y | N | Y | N | Y |
| F | 92 | CURE | MILD | N | N | N | Y | N | N | N | N |
| M | 87 | CURE | MOD | Y | N | Y | | | N | N | Y |
| F | 91 | CURE | MILD | N | Y | Y | N | N | N | N | N |
| F | 60 | CURE | MILD | Y | N | Y | N | N | N | N | N |
| F | 64 | CURE | MILD | Y | Y | Y | N | Y | Y | N | N |
| M | 70 | CURE | MILD | Y | Y | Y | N | N | Y | Y | Y |
| F | 18 | CURE | MILD | Y | N | N | N | N | N | N | N |
| M | 80 | CURE | MOD | Y | N | Y | N | Y | Y | N | Y |
| M | 83 | CURE | MILD | N | N | N | N | N | Y | Y | Y |
| M | 47 | CURE | MILD | N | N | N | Y | N | N | N | N |
| F | 41 | CURE | MILD | N | N | N | Y | N | N | N | N |
| M | 71 | CURE | MOD | Y | Y | Y | N | Y | Y | N | Y |
| F | 80 | FATIGUE | MOD | Y | N | Y | N | Y | Y | Y | Y |
| F | 17 | CURE | MILD | N | N | N | N | N | N | N | N |
| F | 50 | CURE | MILD | Y | Y | N | Y | N | N | N | N |
| M | 32 | CURE | MOD | Y | Y | Y | N | Y | Y | Y | Y |
| F | 55 | CURE | SEVERE | Y | Y | Y | N | N | Y | N | Y |
| F | 66 | CURE | MILD | Y | Y | Y | N | N | N | N | N |
| F | 73 | CURE | MILD | Y | N | Y | N | Y | Y | Y | Y |
| F | 70 | CURE | MILD | Y | N | Y | N | N | Y | Y | Y |
| M | 23 | CURE | MOD | Y | Y | Y | N | Y | N | N | N |
| F | 75 | CURE | MOD | Y | Y | Y | Y | Y | Y | N | Y |
| F | 79 | CURE | MOD | Y | Y | Y | N | Y | Y | N | Y |
| M | 89 | CURE | MOD | Y | N | Y | N | Y | N | Y | Y |
| M | 21 | CURE | MILD | Y | N | Y | N | Y | Y | N | N |
| F | 69 | CURE | SEVERE | Y | Y | Y | N | Y | Y | Y | Y |
| F | 67 | CURE | MILD | N | N | N | Y | N | N | Y | Y |
| M | 55 | CURE | MILD | Y | N | N | N | Y | Y | Y | Y |
| M | 58 | POLYPS | MOD | Y | Y | Y | Y | Y | N | N | Y |
| F | 22 | CURE | MILD | N | N | N | Y | Y | Y | N | N |
| F | 21 | CURE | MILD | Y | N | Y | Y | Y | N | N | N |
| F | 55 | CURE | MILD | Y | Y | N | Y | N | N | N | N |
| F | 26 | CURE | MILD | Y | Y | N | N | N | N | N | N |
| F | 56 | CURE | MILD | N | N | N | N | N | N | N | N |
| F | 90 | CURE | MOD | Y | Y | Y | N | Y | Y | N | Y |
| F | 83 | CURE | MILD | Y | N | Y | N | N | N | N | Y |
| F | 29 | CURE | MILD | Y | N | Y | N | N | N | N | N |
| F | 23 | CURE | MILD | Y | N | Y | N | Y | N | N | N |
| F | 8 | CURE | MILD | N | N | N | N | Y | N | N | N |

Data Summary

| | | | | | | | | | |
|---|---|---|---|---|---|---|---|---|---|
| N = 53 | MOD 17 | Y 40 | Y 21 | Y 35 | Y 16 | Y 28 | Y 22 | Y 10 | T 24 |
| M 21 | MILD 34 | N 13 | N 32 | N 18 | N 37 | N 25 | N 31 | N 43 | N 29 |
| F 32 | SEVERE 2 | | | | | | | | |

Conclusion: Presently, one cornerstone in the COVID-19 treatment paradigm lies in the effective attenuation of inflammation elicited by the virus. Levocetirizine and montelukast, unlike many single target therapeutics, safely attenuate not only histamine and leukotriene D4, respectively, but also synergistically mitigate inflammation across a spectrum of signaling proteins, cell adhesion molecules, and leucocytes: NF-kB, ICAM-1, VCAM-1, IL-4, IL-6, IL-8, RANTES, GM-CSF, TLR-3, AP-1, and eosinophil and neutrophil quantity and migration. Moreover, both molecules in the United States are considered Pregnancy Category B and underscored by millions of days of patient use (montelukast 1998 FDA approval; levocetirizine 2007 FDA approval).

As new COVID variants evolve in a global environment, one of many attributes of the repurposed combination lies in the ability to target cellular protein activity in contrast to viral proteins, an effect not likely to be negated by mutations in the virus genome. Levocetirizine and montelukast appear to offer a significant addition to the treatment of COVID-19, effectively mitigating symptoms without creating concurrent host toxicity. Cumulative data to date suggests the uniquely synergistic combination may reduce the progression and duration as well as prevent/treat many of the aspects of ‘Long COVID,’ thereby cost-effectively reducing both the morbidity and mortality associated with the disease.

What is claimed is:

1. A method of treating a patient suffering from coronavirus disease, the method comprising administering to the patient an effective amount of a combination of levocetirizine and montelukast.

2. The method of claim 1, wherein the treatment causes a decrease in severity in the signs or symptoms of coronavirus disease, including but not limited to one or more of fever or chills, cough, shortness of breath or difficulty breathing, fatigue, muscle or body aches, headache, new loss of taste or smell, sore throat, congestion or runny nose, nausea or vomiting, and/or diarrhea.

3. The method of claim 1, wherein the combination of levocetirizine and montelukast is administered in a sequential manner.

4. The method of claim 1, wherein the combination of levocetirizine and montelukast is administered in a substantially simultaneous manner.

5. The method of claim 1, wherein the combination is administered to the patient by one or more of the routes consisting of enteral, intravenous, intraperitoneal, inhalation, intramuscular, subcutaneous and oral.

6. The method of claim 1, wherein the levocetirizine and montelukast are administered by the same route.

7. The method of claim 1, wherein the levocetirizine and montelukast are administered via different routes.

8. The method of claim 1, wherein one or more of levocetirizine or montelukast are provided as a slow release composition.

9. The method of claim 1, wherein the combination further comprises other medications known for use in treating complications associated with coronavirus disease.

10. The method of claim 1, wherein the combination further comprises a steroid.

11. A method of treating a patient having a symptoms of coronavirus disease, the method comprising administering to the patient an effective amount of a combination of levocetirizine and montelukast.

12. A composition for use in treating a patient having coronavirus disease, the composition comprising a combination of levocetirizine and montelukast.
